(19) United States
(12) Patent Application Publication
(10) Pub. No.: US 2004/0236315 A1
(43) Pub. Date: Nov. 25, 2004

(54) Preoperative surgery site stamp system

(76) Inventor: Robert W. Hered, Atlantic Beach, FL (US)

Correspondence Address: J. Michael Martinez de Andino, HUNTON & WILLIAMS, Riverfront Plaza, East Tower, 951 East Byrd Street, Richmond, VA, 23219-4074, US

(21) Appl. No.: 10/444,632

(22) Filed: May 23, 2003

(51) Int. Cl. A61B 17/00

(52) U.S. Cl. 606/1

(57) ABSTRACT

A method of stamping a patient's body to convey medical information and a kit for accomplishing the method is disclosed. The method provides for positioning medical information related to a medical procedure to be performed at a location on the patient's body related to the procedure to be performed.

### BACKGROUND OF THE INVENTION

1. Field of the Invention

The present invention relates generally to a method of stamping a patient's body to convey medical information and more particularly in certain embodiments to a method of stamping information related to a medical procedure to be performed at a location on the patient's body related to the procedure to be performed. The invention further includes a kit for accomplishing the method.

2. Description of Related Art

Wrong site surgery is a recognized medical problem. Agencies such as the Joint Commission on Accreditation of Healthcare Organizations and Agency for Healthcare Research and Quality have recently considered this problem and found that an effective system for marking or identifying the correct site for surgery is needed. Medical facilities have developed various marking procedures. However, there is no consistent system of marking location of surgery on surgical patients and many of the presently used methods suffer from legibility deficiencies, reliability issues, and/or inconsistency deficiencies. For example, handwritten markings suffer the deficiencies of variation from individual to individual making the markings, and potential illegibility related to the marking skills of the individual making the marking, potential inconsistencies in the kind of mark made for a given piece of information, for example. Stickers have also been used for surgical site marking, but this approach is of questionable reliability because stickers may be easily removed or relocated. Relocation of stickers is of particular concern with children.

Other systems such as temporary tattoo systems have been proposed. (See: Berryman, et al. U.S. Patent Application 2002/0114764A1 and 2002/0164285A1) However, applying tattoos while retaining the integrity of the tattoo is often problematic. Additionally, applying tattoos to some body sites such as the face of an uncooperative young child may be extremely difficult if not impossible. Further, the ability to remove temporary tattoos, make them susceptible to removal or defacing during surgical preparation procedures and/or to inadvertent removal or defacing as a result of patient movements.

The use of external markings have been disclosed for marking the position of indicated treatment on a patient. For example, U.S. Pat. No. 5,407,440 to Zinreich, et al. discloses marking a position on a patient for radiation treatment, U.S. Pat. No. 4,892,096 discloses a method of marking a position on a patient's breast, and U.S. Pat. No. 4,944,737 to Bloom discloses a surgical stencil with perforations allowing for skin to be marked for skin flap surgery. These marking systems convey information related to a body position, but none of these disclosed methods convey patient information that affirmatively indicates a site at which a procedure is to be performed or alternatively a site which is not to be treated.

Despite these disclosures, a need remains for a method and/or system which conveniently and consistently identifies the site to be subjected to surgery during the preparation process for the surgery and has legibility at the time of surgery.

### SUMMARY OF THE INVENTION

The present invention includes a method for conveying medical procedure patient information. The method includes identifying a selected patient information, selecting a stamp that conveys the selected patient information and which has a stamp surface treated with an ink, and applying the stamp surface at a region on the patient to be subjected to a medical procedure. The selected patient information relates to the region on the patient. The stamp may convey information regarding a surgical procedure such as identification of the location on a patient's body where an identified medical procedure is to be performed.

A method is also provided for conveying patient information. The method includes identifying a selected patient information; selecting a stamp that conveys the selected patient information which has a stamp surface treated with an ink, and applying the stamp surface to the patient to be subjected to medical procedure.

The stamp of the invention may include verbiage, a schematic depiction or a combination of verbiage and schematic depiction.

The ink at the surface of the stamp is compatible with application to a human body and, in certain embodiments of the invention, it is preferable that the ink is resistant to removal under surgical preparation conditions.

Further, a kit is provided. The kit includes a container defining an enclosure of predefined dimensions, at least one stamp including predefined patient information positioned within the enclosure and an ink supply wherein the ink is compatible with application to a human body positioned within the enclosure. The enclosure may be sealed. Further, the sealed enclosure may be sterilized. The ink supply may be an ink pad or an ink supply positioned at the surface of the stamp. In certain embodiments it is desirable that the ink of the ink supply is resistant to removal under surgical preparation conditions.

### BRIEF DESCRIPTION OF THE DRAWINGS

FIG. 1 shows an embodiment of a stamp that conveys patient information;

FIG. 2 shows an embodiment of a stamp surface that conveys patient information using a combination of verbiage and schematic depiction;

FIG. 3 shows an embodiment of a stamp surface that conveys patient information related to the position of a medical procedure;

FIG. 4 shows an embodiment of a stamp surface that conveys patient information; and

FIG. 5 shows an embodiment of a stamp grasping portion having a marking to facilitate stamp orientation.

### DETAILED DESCRIPTION OF THE INVENTION

The present invention involves a method for conveying patient information related to a medical procedure using a stamp system. The method may be used for patients of any age and is particularity well suited for use with infants or young children in which body areas designated for a medical procedure may be small and/or for patients who are not particularly cooperative either intentionally or inadvertently with medical personnel. A kit suitable for performing the method is also described.

In an exemplary embodiment of the invention the method includes: identifying a selected patient information, selecting a stamp that conveys the selected patient information and has a stamp surface treated with ink and applying the stamp surface treated with ink to a patient to be subjected to a medical procedure.

The patient information may include indication of the region on the patient where the procedure is to be performed; such as “surgical site” or “YES”, indication of a region on a patient where a procedure should not be performed, such as “do not operate here”, or “NO”, or an indication of patient specific data such as blood type, tissue type, allergies to certain medications and the like, for example.

In some embodiments of the invention the patient information relates specifically to the region on a patient's body which will be subjected to the medical procedure or alternatively to a region of the patient's body which should not be subjected to the medical procedure. Such patient information facilitates avoiding wrong site surgery. In other embodiments the information may facilitate correct identification of a site on a patient's body for treatment or testing, or the like, for example.

In some embodiments the patient information may include critical patient specific information which facilitates avoiding such unfortunate medical accidents such as transfusing an incompatible blood type to a patient, administering a drug or medication to which the patient is allergic, or failure to recognize a known medical condition which renders the patient in a compromised condition, for example. As one skilled in the art will appreciate, the selected patient information may be a single unit of patient information or multiple units of patient information. For example, the selected information may include a single unit of patient information regarding the location of a medical procedure relative to the patient body or both information regarding the location of medical procedure relative to the patient's body and the patient's blood type, for example. This simple example is provided for illustration purposes and as one skilled in the art will appreciate numerous types of units of patient information and/or combination of units of patient information may be used in the method of the invention.

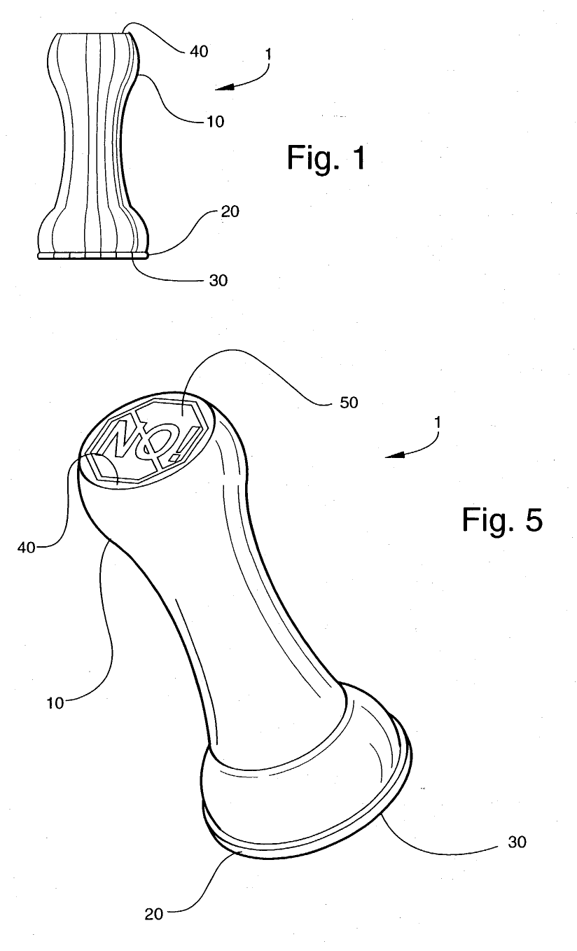

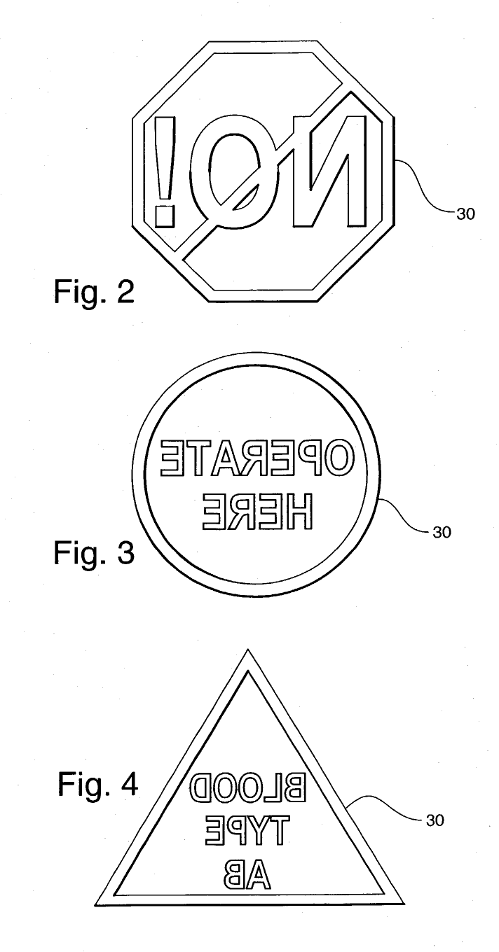

In one embodiment, the stamp of the invention is a stamp with the general features of stamping devices well known in the art. As shown in FIG. 1, the stamp 1 has a grasping portion 10 with a top 40, an imprint portion 20, and the imprint portion 20 has a stamp surface 30. The grasping portion 10 provides a region which the user of the stamp 1, typically medical personnel, can grasp when applying the stamp to a body region of a patient. The imprint portion 20 is affixed to the grasping portion 10. The imprint portion 20 has an exposed stamp surface 30. The imprint portion 20 comprises at least one region that extends beyond at least one other region of the imprint portion 20 such that a pattern is formed that creates verbiage, a pictorial depiction or a combination thereof that relates to identified patient information. In some embodiments the perimeter of the stamp surface 30 may have a specified geometric shape such as octagon or triangle. The shape may be defined to relay general information such as stop or caution, for example, and thereby, provide visual warning signals to medical personnel in addition to the patient information. FIGS. 2-4 show examples of a stamp 1 as viewed at the stamp surface 30.

Optionally, the stamp 1 may include an orientation marking 50 on the top 40 as shown in FIG. 5. This marking facilitates orientation of the stamp portion 20 immediately prior to stamping the patient's body region. The orientation marking 50 may, for example, replicate the image or verbiage of the imprint portion 20 as shown in FIG. 5. Alternatively, it may be a marking that indicates orientation such as a “this side up” marking and/or an arrow pointing upward marking, for example. As one skilled in the art will appreciate numerous types of images, words or combination thereof may be used for the orientation marking 50 to indicate orientation.

Referring again to FIG. 1, the gripping portion 10 and imprint portion 20 of the stamp 1 may be formed as a single unit such as in a molding process for example. Alternatively, the gripping portion 10 and the imprint portion 20 may be formed separately and fixedly or removably joined by methods known in the art.

The stamp surface 30 of the imprint portion 20 is treated with ink prior to applying the stamp 1 to the patient's body. The ink may be applied using an ink pad immediately prior to use of the stamp 1 as is well known in the art. Alternatively, the stamp 1 may be pretreated with ink. In embodiments employing pre-inked stamps, it may be desirable to provide a protective cover over the inked stamp surface 30 which can be removed immediately prior to use.

The ink should be compatible with use on a human body. For example, it should be visible on human skin, not pose a toxicological danger, and the like. In some embodiments it is desirable that the ink be resistant to surgical preparation procedures. This facilitates application of the stamp prior to the medical procedures with legibility maintained at least until the procedure is begun. Gentian violet dye is one example of an ink that may be suitable for such purposes.

In some embodiments it may be desirable to use at least a pair of stamps one indicating affirmatively where the procedure should be performed and a second stamp indicating where the procedure should not be performed. In such embodiments it may be desirable to use color coded inks in addition to stamps with imprint surfaces reflecting the patient information. For example, a negative patient information stamp such as a “do not operate here” stamp may be coated on the stamp surface 30 with red ink while an affirmative “operate here” stamp that identifies the site of the procedure may be coated on the stamp surface 30 with a green ink. As one skilled in the art will appreciate many combinations of ink color and stamp messages are possible. While the patient information is conveyed by the stamp selected, establishing a color coding system which reflects a known relationship between the color code and the patient information on the stamp provides a further readily identifiable marker for conveying patient information to the personnel performing the medical procedure.

The stamp surface 30 treated with ink is applied to the patient's body. In some embodiments the patient's body region selected for application of the stamp surface 30 is directly related to the selected patient information conveyed by the stamp such as “operate here” with the stamp being positioned on the site of the medical procedure, for example on a limb or the brow above an eye designated for the operation or like. In other embodiments, the stamp may be placed in a visible or predetermined area. For example a stamp indicating blood type might be placed in a prominent location such as on the forehead or chest or on an arm where a transfusion might be given in addition to and/or alternatively to placing the stamp at the site of the medical procedure.

In one embodiment a kit is provided. The kit comprises a container defining an enclosure of a predefined dimension, a stamp including predefined patient information positioned within the enclosure, and an ink supply including ink that is compatible with application to a human body positioned within the enclosure.

This kit is suitable for use in performing the method of the invention. In some embodiments the enclosure may be sealed to maintain cleanliness of the stamp and ink. In some embodiments it may be desirable to seal the container and provide a sterilized environment within the sealed portion of the container. Sterilization of the sealed enclosure may be accomplished by sterilization methods known in the art. The ink supply may be an ink pad as is known in the art, for example. Alternatively, the ink supply may be pre-applied to the stamp. In embodiments where the ink supply is pre-applied to the stamp it may be desirable to further provide a removalable protective cover over the ink that protects the ink supply prior to use and that can be readily removed at the time of use.

As one skilled in the art will appreciate the method and kit of the present invention are well suited for use in developing a standardized system for marking surgical sites. Once a standard series of markings is defined, stamps may be designed for identified standard markings, and numerous exact replicate stamps produced readily and at low cost. The kits of the invention are amendable to mass production at production costs which render them disposable or single use kits. Single use stamps may be desirable in some embodiments, to minimize transmission of potential containments.

The method is equally applicable for facilitating consistent markings within a given medical facility or for a universal marking system to be used broadly within multiple facilities. The method is well suited for use in establishing and implementing a national standardized system of marking patients for surgery.

Further, the method of the invention facilitates reduction of legibility problems associated with marking patients for surgery. This is accomplished in part by eliminating handwritten markings on a patient. Handwriting is subject to individual variation. Additionally, handwriting requires multiple motions to apply a notation to a position and is subject to space constraints restricting the amount of information that may be written. Variation for a given type of patient information marking may be significantly reduced or eliminated by the present method as the stamps may be readily replicated creating a supply of identical stamps for a given patient's information with stamp to stamp variation reduced to a minimum. Additionally, a stamp imprint surface can provide an enhanced amount of information relative to the area of the stamp imprint surface as compared to amount of information that can be conveyed by handwriting in the same report. This enhancement of information may include facilitating provision of specific patent information on a very small area such as the forehead of an infant for example or providing multiple patient information in a very confined region such as procedure site information and treatment information in a single modestly sized stamp, for example.

The potential for smearing and reduced legibility may also be minimized by the method of the invention. Applying the stamp in a single quick motion facilitates marking young children, and incapacitated persons who may either intentionally or inadvertently fail to cooperate with and/or resist the attempts of medical personnel to make a marking. The single quick motion of applying a stamp provides advantages over applying a temporary tattoo which must be positioned and adhered to the surface of the skin without folding and without gaps where the tattoo is not adhered to the skin.

The method of the invention avoids the inherent difficulty of writing on a human skin which is not a surface well suited for writing. Further, selection of the ink used can minimize irritation to sensitive skin and provide for various levels of robustness of the stamp. For example, an ink may be selected with resistance to surgical preparation procedures. Such ink may retain legibility through surgical preparation procedures and remain legible at the time of surgery. Known, temporary tattoos typically lack this robustness.

The method of the invention avoids the problem of transferability inherent to the use of adhesive stickers for body site markings. Young children have been known to remove stickers and to move body site identification stickers to incorrect sites on their bodies.

Those persons skilled in the art will appreciate that the present invention is susceptible to a broad utility and application. Many embodiments and adaptations of the present invention other than those herein described, as well as many variations and modifications will be apparent from or reasonably suggested by the present invention and the foregoing description thereof, without departing from the substance or scope of the present invention. Accordingly, while the present invention has been described herein in detail in exemplary embodiments, it is to be understood that this disclosure is only illustrative and exemplary of the present invention and is made merely for purposes of providing a full and enabling disclosure of the invention. The foregoing is not intended or to be construed to limit the present invention or otherwise to exclude any such other embodiments, adaptations, variations, modifications, the present invention being limited only by the claims appended hereto and the equivalents thereof.

1. A method for conveying medical procedure patient information comprising:
identifying a selected patient information;
wherein the surface of the stamp has a stamp surface and the stamp surface is treated with an ink; and
applying the stamp surface to a patient at a region on the patient to be subjected to a medical procedure wherein the selected patient information conveyed by the stamp relates to the region on the patient and wherein the stamp includes verbiage.

2. The method of claim 1, wherein the stamp conveys information regarding a surgical procedure.

3. The method of claim 1, wherein the patient information comprises identification of a predetermined location on a patient's body where an identified medical procedure is to be performed.

4. The method of claim 1, wherein the ink at the surface of the stamp is compatible with application to a human body and is resistant to removal under surgical preparation conditions.

5. The method of claim 1, wherein the stamp includes a schematic depiction.

6. (cancelled)

7. A method for conveying patient information comprising:
identifying a selected patient information;
selecting a stamp wherein the stamp conveys the selected patient information and wherein the stamp has a stamp surface and the stamp surface is treated with an ink and wherein the stamp includes verbiage; and
applying the stamp surface to a patient to be subjected to a medical procedure wherein the selected patient information conveyed by the stamp provides selected patient information related to the medical procedure.

8. The method of claim 7, wherein the stamp conveys information regarding a surgical procedure.

9. The method of claim 7, wherein the patient information comprises identification of a predetermined location on a patient's body where an identified medical procedure is to be performed.

10. The method of claim 7, wherein the ink on the surface of the stamp is compatible with application to a human body and is resistant to removal under surgical preparation conditions.

11. The method of claim 7, wherein the stamp includes a schematic depiction.

12. (cancelled)

13. A kit comprising:
container defining an enclosure of predefined dimensions;
at least one stamp including predefined patient information positioned within the enclosure wherein the stamp contains verbiage; and
an ink supply wherein the ink is compatible with application to a human body and the ink supply is positioned within the enclosure.

14. The kit of claim 13, wherein the enclosure of predefined dimension is a sealed enclosure.

15. The kit of claim 14, wherein the sealed enclosure is sterilized.

16. The kit of claim 13, wherein the ink of the ink supply is resistant to removal under surgical preparation conditions.

17. The kit of claim 13, wherein the ink supply is an ink pad.

18. The kit of claim 13, wherein the ink supply is positioned at the surface of the stamp.
